(19) United States
(12) Patent Application Publication
(10) Pub. No.: US 2026/0289402 A1
(43) Pub. Date: Sep. 24, 2026

(54) ELECTRONIC DEVICES AND METHODS FOR FINE-TUNING ARTIFICIAL INTELLIGENCE MODELS THAT OUTPUT MOLECULES FOR PROTEINS

(71) Applicant: POSTECH RESEARCH AND BUSINESS DEVELOPMENT FOUNDATION, Pohang-si (KR)

(72) Inventors: Dongwoo KIM, Pohang-si (KR); Seungbeom LEE, Pohang-si (KR); Munsun JO, Pohang-si (KR)

(21) Appl. No.: 19/465,697

(22) Filed: Jan. 30, 2026

(30) Foreign Application Priority Data
Mar. 21, 2025 (KR) 10-2025-0036570
Jul. 31, 2025 (KR) 10-2025-0104866

(51) Int. Cl. G06N 20/00; G16B 15/30; G16B 40/00

(52) U.S. Cl. CPC G06N 20/00; G16B 15/30; G16B 40/00

(57) ABSTRACT

An electronic device for fine-tuning an artificial intelligence model that outputs molecules for a protein is provided. The electronic device for fine-tuning an artificial intelligence model that outputs molecules for a protein, comprising: a processor; and a memory operatively coupled to the processor, wherein the memory stores instructions that, when executed, cause the processor to: identify a molecule suitable for a target protein by inputting the target protein into the artificial intelligence model, identify a plurality of rewards for each of a plurality of properties required for new drug design, based on the molecule, identify a final reward for the molecule by normalizing each of the plurality of rewards, determine a preference for the molecule based on the final reward, and fine-tune the artificial intelligence model by using a loss function based on the preference.

### CROSS-REFERENCE TO RELATED APPLICATION

This application claims priority under 35 U.S.C § 119 to Korean Patent Application No. 10-2025-0036570 filed on Mar. 21, 2025, and Korean Patent Application No. 10-2025-0104866 filed on Jul. 31, 2025, in the Korean Intellectual Property Office, the entire contents of which are hereby incorporated by reference.

### TECHNICAL FIELD

The disclosure relates to electronic devices and methods for fine-tuning artificial intelligence models that output molecules for proteins.

### BACKGROUND

The content set forth in this section merely provides background information on the present embodiments and does not constitute prior art.

Recently, the use of methods for designing new drugs utilizing artificial intelligence (AI) and deep learning has been increasing. For example, there has been an increasing use of technology for generating optimal molecules (ligands) for target proteins by using structure-based drug design (SBDD) technology, which generates molecules with a three-dimensional structure suitable for the protein's three-dimensional structure, when the protein's three-dimensional structure is given.

Furthermore, recent structure-based drug design has focused on generating molecules with high binding affinity to target proteins. However, for structure-based drug design to be effectively utilized in the actual new drug development process, it is necessary to consider not only the binding affinity between the target protein and the molecule, but also various other properties together. Therefore, there has been a need for a technology that generates molecules suitable for target proteins using an artificial intelligence model by taking into account various properties in new drug design.

### SUMMARY

It is an object of the present disclosure to provide an electronic device and a method for fine-tuning an artificial intelligence model that outputs molecules for proteins, which can optimize a plurality of properties simultaneously.

The objects of the present disclosure are not limited to the object mentioned above, and other objects and advantages of the present disclosure that have not been mentioned can be understood by the following description and will be understood more clearly by the embodiments of the present disclosure. Further, it will be readily appreciated that the objects and advantages of the present disclosure can be realized by the means set forth in the claims and combinations thereof.

According to some embodiments of the present disclosure, an electronic device for fine-tuning an artificial intelligence model that outputs molecules for a protein, comprising: a processor; and a memory operatively coupled to the processor, wherein the memory stores instructions that, when executed, cause the processor to: identify a molecule suitable for a target protein by inputting the target protein into the artificial intelligence model, identify a plurality of rewards for each of a plurality of properties required for new drug design, based on the molecule, identify a final reward for the molecule by normalizing each of the plurality of rewards, determine a preference for the molecule based on the final reward, and fine-tune the artificial intelligence model by using a loss function based on the preference.

According to some embodiments of the present disclosure, wherein the instructions cause the processor to: scale each of the plurality of rewards, identify a plurality of normalized values by normalizing each of the scaled plurality of rewards, and identify the final reward based on the plurality of normalized values.

According to some embodiments of the present disclosure, wherein the instructions cause the processor to normalize each of the plurality of rewards by using softmax-based normalization.

According to some embodiments of the present disclosure, wherein the instructions cause the processor to: identify a mean value for the plurality of normalized values, and identify the final reward based on the plurality of normalized values and the mean value.

According to some embodiments of the present disclosure, wherein the instructions cause the processor to identify the final reward based on the plurality of normalized values and the mean value by using uncertainty regularization.

According to some embodiments of the present disclosure, wherein the instructions cause the processor to identify the final reward based on a deviation between the plurality of normalized values and the mean value.

According to some embodiments of the present disclosure, wherein the instructions cause the processor to: receive the molecule suitable for the target protein and a second molecule from the artificial intelligence model, and determine the preference for the molecule by comparing a second final reward for the second molecule with the final reward for the molecule.

According to some embodiments of the present disclosure, wherein the plurality of properties comprises at least one of binding affinity between the target protein and the molecule, effectiveness of the molecule, synthetic accessibility, and drug-likeness.

According to some embodiments of the present disclosure, an electronic device for fine-tuning an artificial intelligence model that outputs molecules for a protein, comprising: a processor; and a memory operatively coupled to the processor, wherein the memory stores instructions that, when executed, cause the processor to: identify a molecule suitable for a target protein by inputting the target protein into the artificial intelligence model, identify a plurality of rewards for each of a plurality of properties required for new drug design, based on the molecule, identify a final reward for the molecule by normalizing each of the plurality of rewards, fine-tune the artificial intelligence model based on the final reward.

According to some embodiments of the present disclosure, wherein the instructions cause the processor to: scale each of the plurality of rewards, identify a plurality of normalized values by normalizing each of the scaled plurality of rewards, and identify the final reward based on the plurality of normalized values.

According to some embodiments of the present disclosure, wherein the instructions cause the processor to normalize each of the plurality of rewards by using softmax-based normalization.

According to some embodiments of the present disclosure, wherein the instructions cause the processor to: identify a mean value for the plurality of normalized values, and identify the final reward based on the plurality of normalized values and the mean value.

According to some embodiments of the present disclosure, wherein the instructions cause the processor to: determine a preference for the molecule based on the final reward, and fine-tune the artificial intelligence model by using a loss function based on the preference.

According to some embodiments of the present disclosure, wherein the instructions cause the processor to: receive the molecule suitable for the target protein and a second molecule from the artificial intelligence model, and determine the preference for the molecule by comparing a second final reward for the second molecule with the final reward for the molecule.

According to some embodiments of the present disclosure, a method of fine-tuning an artificial intelligence model that outputs molecules for a protein, performed by an electronic device, comprising the steps of: identifying a molecule suitable for a target protein by inputting the target protein into the artificial intelligence model; identifying a plurality of rewards for each of a plurality of properties required for new drug design, based on the molecule; identifying a final reward for the molecule by normalizing each of the plurality of rewards; determining a preference for the molecule based on the final reward; and fine-tuning the artificial intelligence model by using a loss function based on the preference.

According to some embodiments of the present disclosure, wherein the step of identifying the final reward for the molecule comprises the steps of: scaling each of the plurality of rewards; identifying a plurality of normalized values by normalizing each of the scaled plurality of rewards; and identifying the final reward based on the plurality of normalized values.

According to some embodiments of the present disclosure, wherein the step of identifying the plurality of normalized values normalizes each of the plurality of rewards by using softmax-based normalization.

According to some embodiments of the present disclosure, wherein the step of identifying the final reward based on the plurality of normalized values comprises the steps of: identifying a mean value for the plurality of normalized values; and identifying the final reward based on the plurality of normalized values and the mean value.

According to some embodiments of the present disclosure, wherein the step of identifying the final reward based on the plurality of normalized values and the mean value identifies the final reward based on the plurality of normalized values and the mean value by using uncertainty regularization.

According to some embodiments of the present disclosure, wherein the step of determining the preference for the molecule based on the final reward comprises the steps of: receiving the molecule suitable for the target protein and a second molecule from the artificial intelligence model; and determining the preference for the molecule by comparing a second final reward for the second molecule with the final reward for the molecule.

The electronic device and method for fine-tuning an artificial intelligence model that outputs molecules for proteins of the present disclosure can generate molecules with properties suitable for drug development by optimizing a plurality of properties and generating molecules for a target protein.

Furthermore, the electronic device and method for fine-tuning an artificial intelligence model that outputs molecules for proteins of the present disclosure can reduce the time and cost required for new drug development by optimizing a plurality of properties and generating molecules for a target protein.

In addition to the foregoing description, specific effects of the present disclosure will be stated together while describing specific details for implementing the present disclosure below.

### BRIEF DESCRIPTION OF THE DRAWINGS

FIG. 1 is a diagram for describing an electronic device for fine-tuning an artificial intelligence model that outputs molecules for a protein in accordance with an embodiment of the present disclosure.

FIG. 2 is a diagram for describing an electronic device for fine-tuning an artificial intelligence model that outputs molecules for a protein and the artificial intelligence model in accordance with an embodiment of the present disclosure.

FIG. 3 is a diagram for describing the operation of the electronic device (e.g., the processor) of FIGS. 1 and 2.

FIG. 4 is a diagram for describing step S300 of FIG. 3.

FIG. 5 shows a mathematical formula for describing step S320 of FIG. 4.

FIG. 6 shows a mathematical formula for describing step S330 of FIG. 4.

FIG. 7 shows a mathematical formula for describing step S500 of FIG. 3.

### DETAILED DESCRIPTION OF THE PREFERRED EMBODIMENTS

The terms or words used in the present specification and the claims should not be interpreted as being limited to their general or dictionary meanings. In accordance with the principle that the inventor may define the concepts of terms or words to best describe his or her invention, they should be interpreted based on the meanings and concepts consistent with the technical idea of the present invention. Furthermore, the embodiments described in the present specification and the configurations illustrated in the drawings are merely examples for realizing the invention and do not represent the entirety of the technical idea of the present invention. It should be understood that, at the time of filing, there may be various equivalents, modifications, and applicable examples that can replace these embodiments.

The terms such as “first,” “second,” “A,” “B,” and the like, used in the present specification and the claims, may be used to describe various components, but such components should not be limited by these terms. These terms are merely used to distinguish one component from another. For example, without departing from the scope of the present invention, a first component may be referred to as a second component, and similarly, the second component may be referred to as the first component. The term “and/or” includes any combination of the related listed items or any one of the related listed items.

The terms used in the present specification and the claims are used merely to describe particular embodiments and are not intended to limit the invention. Unless clearly indicated otherwise by the context, singular expressions include plural expressions. In this application, the terms such as “include” or “have” should be understood as indicating the presence or possibility of addition of features, numbers, steps, operations, elements, parts, or combinations thereof described in the specification, without precluding the existence or addition of one or more other features, numbers, steps, operations, elements, parts, or combinations thereof.

Unless otherwise defined, all terms used herein, including technical or scientific terms, have the same meanings as those generally understood by a person of ordinary skill in the technical field to which the present invention pertains.

Terms defined in commonly used dictionaries should be interpreted as having meanings consistent with their meanings in the relevant technical context, and unless explicitly defined in this application, should not be interpreted in an idealized or excessively formal sense. Furthermore, each structure, process, procedure, or method included in the embodiments of the present invention may be shared to the extent that they do not technically contradict each other.

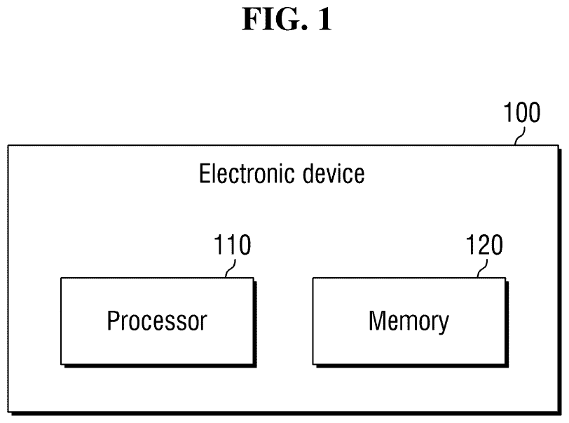

Hereinafter, an electronic device and a method for fine-tuning an artificial intelligence model that outputs molecules for a protein in accordance with an embodiment of the present disclosure will be described with reference to FIGS. 1 to 7.

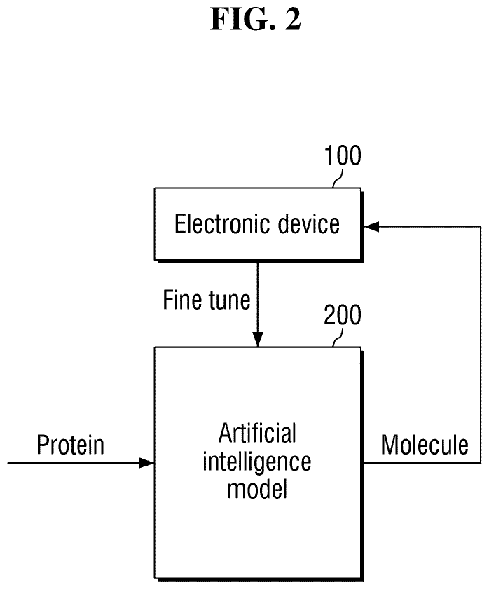

FIG. 1 is a diagram for describing an electronic device for fine-tuning an artificial intelligence model that outputs molecules for a protein in accordance with an embodiment of the present disclosure. FIG. 2 is a diagram for describing an electronic device for fine-tuning an artificial intelligence model that outputs molecules for a protein and the artificial intelligence model in accordance with an embodiment of the present disclosure.

Referring to FIG. 1, an electronic device (hereinafter, “electronic device”) 100 for fine-tuning an artificial intelligence model that outputs molecules for a protein may include a processor 110 and a memory 120.

The electronic device 100 may communicate with an artificial intelligence model 200. The electronic device 100 may be a device that fine-tunes the artificial intelligence model 200. The artificial intelligence model 200 may be a model that receives a protein as input and generates a molecule suitable for the input target protein. The molecule generated by the artificial intelligence model 200 may be a drug molecule used for new drug development. The artificial intelligence model 200, fine-tuned by the electronic device 100, can generate a molecule that satisfies a plurality of properties of the target protein (e.g., binding affinity between the target protein and the molecule, effectiveness of the molecular structure, synthetic accessibility, quantitative estimate of drug likeness, etc.). The artificial intelligence model 200 may be, for example, one of TargetDiff, MolCraft, IPDiff, and AliDiff. However, the artificial intelligence model 200 is not limited thereto, and may be a model that receives proteins as input and outputs molecules used in new drug development.

One or more other components (e.g., a communication module) may be added to the electronic device 100. In some embodiments, some of these components may be implemented in a single integrated circuit.

The memory 120 may store various data used by at least one component (e.g., the processor 110) of the electronic device 100. The data may include, for example, software (e.g., programs) and input or output data for commands associated therewith. The memory 120 may include volatile memory or non-volatile memory.

The memory 120 may store commands, information, or data associated with the operation of the components included in the electronic device 100. For example, the memory 120 may store instructions that, when executed, enable the processor 110 to perform the various operations described herein.

The processor 110 may be operatively coupled to the memory 120 in order to perform the overall functions of the electronic device 100. The processor 110 may include, for example, one or more processors. The one or more processors may include, for example, an image signal processor (ISP), an application processor (AP), or a communication processor (CP).

The processor 110 may control at least one other component (e.g., a hardware or software component) of the electronic device 100 coupled to the processor 110 by executing, for example, software (e.g., a program), and perform various data processing or calculations. According to one embodiment, as at least part of the data processing or calculations, the processor 110 may load commands or data received from other components (e.g., a communication module) into the memory 120, process the commands or data stored in the memory 120, and store the resulting data in the memory 120. According to one embodiment, the processor 110 may include a main processor (e.g., a central processing unit or application processor) and an auxiliary processor (e.g., a graphics processing unit, image signal processor, sensor hub processor, or communication processor) that can operate independently or together with the main processor. Additionally or alternatively, the auxiliary processor may be set to use less power than the main processor or to be specialized for specified functions. The auxiliary processor may be implemented separately from or as part of the main processor. The program may be stored as software in the memory 120 and may include, for example, an operating system, middleware, or an application.

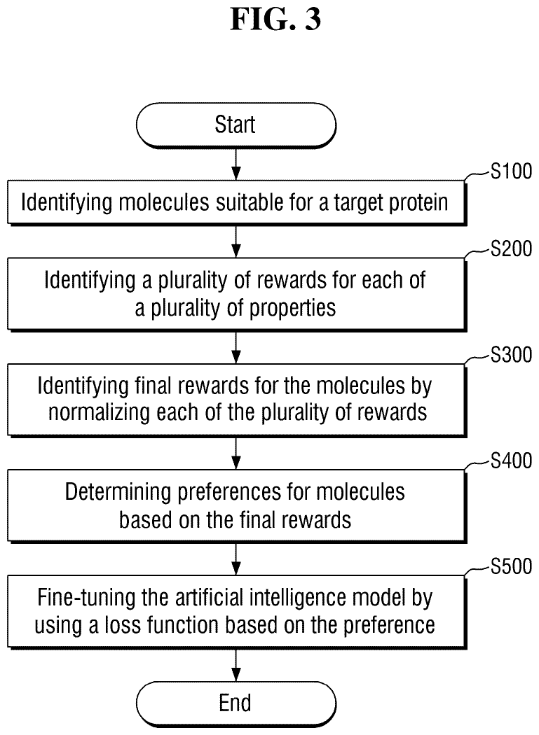

FIG. 3 is a diagram for describing the operation of the electronic device (e.g., the processor) of FIGS. 1 and 2. In the following, it is assumed that the processor 110 of the electronic device 100 in FIGS. 1 and 2 performs the process of FIG. 3. The operations described as being performed by the processor 110 of the electronic device 100 may be implemented as instructions that can be performed (or executed) by the processor 110 of the electronic device 100. The instructions may be stored, for example, on a computer recording medium or in the memory 120 of the electronic device 100 of FIGS. 1 and 2.

Referring to FIG. 3, in a method for fine-tuning an artificial intelligence model that outputs molecules for a protein, the processor 110 can identify molecules suitable for a target protein (S100). The processor 110 can input the target protein into the artificial intelligence model 200 prior to fine-tuning, and can identify molecules suitable for the target protein from the artificial intelligence model 200.

In the method for fine-tuning an artificial intelligence model that outputs molecules for a protein, the processor 110 can identify a plurality of rewards for each of a plurality of properties (S200).

The plurality of properties may be properties of a molecule (ligand) required for new drug design. The plurality of properties may include, for example, at least two of the binding affinity between the target protein and the molecule, effectiveness of a molecular structure, synthetic accessibility, and quantitative estimate of drug likeness (QED). The binding affinity may refer to the binding affinity between the target protein and the molecule. The effectiveness of a molecular structure may mean that the molecule output by the artificial intelligence model 200 has a stable structure in three-dimensional space, and its chemical energy is stable. The effectiveness of a molecular structure may mean that a molecule has a physically and chemically stable structure. The synthetic accessibility and quantitative estimate of drug likeness may be properties required to be developed as a new drug.

The plurality of rewards for each of the plurality of properties may be obtained via a prediction model (a deep learning-based model), an appropriate function, and/or simulation. The prediction model may be, for example, a docking score and binding affinity prediction model, a Tox21 and Ames test-based prediction model, a QED score, a logS prediction model, etc. The processor 110 can derive the plurality of rewards by using the same or different prediction models for each of the plurality of properties.

In the method for fine-tuning an artificial intelligence model that outputs molecules for a protein, the processor 110 can identify final rewards for the molecules by normalizing each of the plurality of rewards (S300).

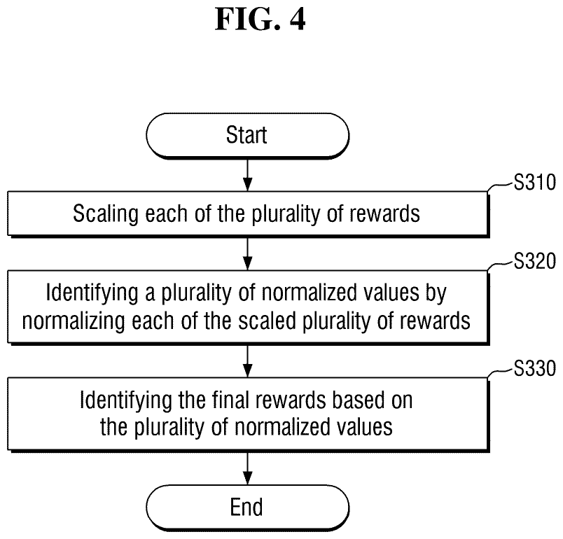

FIG. 4 is a diagram for describing step S300 of FIG. 3.

Referring to FIG. 4, in the method for fine-tuning an artificial intelligence model that outputs molecules for a protein, the processor 110 can first scale each of the plurality of rewards in order to identify the final rewards for the molecules (S310). Since each of the plurality of rewards has a different scale, scaling is necessary for each of the plurality of rewards.

The processor 110 can scale each of the plurality of rewards, for example, by using the rewards for the generated molecules and a parameter for adjusting the scale of the reward.

In the method for fine-tuning an artificial intelligence model that outputs molecules for a protein, the processor 110 can identify a plurality of normalized values by normalizing each of the scaled plurality of rewards (S320). The processor 110 can normalize each of the scaled plurality of rewards using, for example, softmax-based normalization, thereby deriving a plurality of normalized values for each of the scaled plurality of rewards.

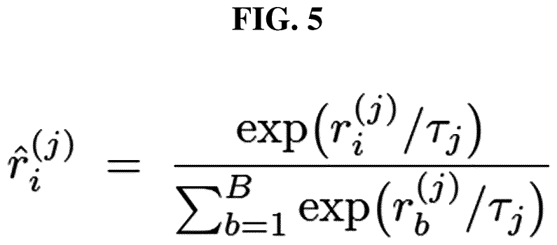

FIG. 5 shows a mathematical formula for describing step S320 of FIG. 4. The mathematical formula in FIG. 5 may be a mathematical formula for deriving a plurality of normalized values for each of the scaled plurality of rewards.

The plurality of normalized values

$${\hat{r}}_{i}^{( j )}$$

can be derived based on a reward

$$exp ( {r}_{i}^{( j )} / {τ}_{j} )$$

scaled based on a reward

$${r}_{i}^{( j )}$$

for the j-th property of the plurality of properties for the i-th molecule and a scaling parameter τⱼ, and the sum of rewards

$$exp ( {r}_{b}^{( j )} / {τ}_{j} )$$

scaled for all molecules used to derive the normalized values. In the mathematical formula of FIG. 5, b may be the index of each molecule in the batch, and B may be the size of the batch.

For example, when normalizing rewards for a first property for a first molecule and a second molecule generated by the artificial intelligence model 200 for a target protein, the processor 110 can identify a normalized value for the first property of the first molecule using the mathematical formula in FIG. 5, based on the sum of a reward value scaled for the first property of the first molecule and a reward value scaled for the first property of the second molecule, and the reward value scaled for the first property of the first molecule. Further, the processor 110 can identify a normalized value for the first property of the second molecule using the mathematical formula in FIG. 5, based on the sum of the reward value scaled for the first property of the first molecule and the reward value scaled for the first property of the second molecule, and the reward value scaled for the first property of the second molecule.

Referring again to FIG. 4, in the method for fine-tuning an artificial intelligence model that outputs molecules for a protein, the processor 110 can identify final rewards based on the plurality of normalized values (S330). The processor 110 can derive a final reward for a plurality of properties of a single molecule.

For example, for the first molecule and the second molecule generated by the artificial intelligence model 200 for the target protein, the processor 110 can identify a final reward based on the plurality of normalized values for the first molecule, and can identify a final reward based on the plurality of normalized values for the second molecule.

Since the final reward is derived based on the plurality of normalized values, and the plurality of normalized values are values derived based on rewards for each of the plurality of properties, the final reward may be a single value that includes values for the plurality of properties.

The processor 110 can identify the final reward based on the plurality of normalized values and a mean value, for example, using uncertainty regularization.

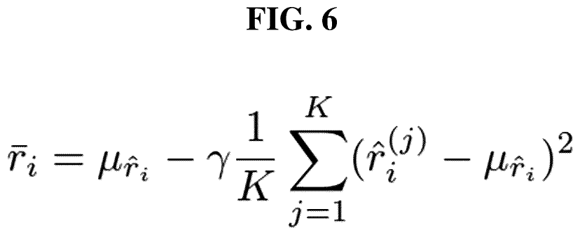

FIG. 6 shows a mathematical formula for describing step S330 of FIG. 4. The mathematical formula in FIG. 6 may be a mathematical formula for deriving a final reward for a molecule based on a plurality of normalized values derived for the molecule.

The processor 110 can identify a mean value μ_{\hat{r}}_{ᵢ}, for the plurality of normalized values

$${\hat{r}}_{i}^{( j )} .$$

Each of the plurality of normalized values

$${\hat{r}}_{i}^{( j )} .$$

may be a normalized value for a j-th property of the plurality of properties for the i-th molecule. In the mathematical formula of FIG. 6, γ is a hyperparameter (e.g., γ≥0) that adjusts the variance penalty, and K may be the number of the plurality of properties.

The processor 110 can identify the final reward r̅ᵢ based on the deviauon

$$( {\hat{r}}_{i}^{( j )} - {μ}_{{\hat{r}}_{i}} )$$

between the plurality of normalized values

$${\hat{r}}_{i}^{( j )}$$

and the mean value μ_{\hat{r}}_{ᵢ}, for example, using the mathematical formula of FIG. 6.

Referring again to FIG. 3, in the method for fine-tuning an artificial intelligence model that outputs molecules for a protein, the processor 110 can determine preferences for molecules based on the final rewards (S400).

The processor 110 can, for example, compare the final rewards for each of at least two molecules generated for the target protein with each other, thereby determining a preference for each of the molecules. The preference can be determined relatively by comparing the final rewards for each of at least two molecules generated for the target protein.

For example, by comparing a first final reward for a first molecule and a second final reward for a second molecule with each other, the processor 110 can identify the first molecule with the larger final reward as a preferred molecule for the target protein, and identify the second molecule with the smaller final reward as a non-preferred molecule for the target protein.

In the method for fine-tuning an artificial intelligence model that outputs molecules for a protein, the processor 110 can fine-tune the artificial intelligence model by using a loss function based on the preference (S500).

The loss function based on the preference may be a loss function that reflects causing the artificial intelligence model 200 to generate preferred molecules with a higher probability than non-preferred molecules.

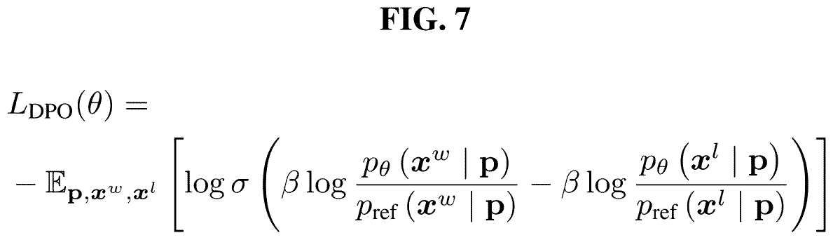

FIG. 7 shows a mathematical formula for describing step S500 of FIG. 3. The mathematical formula of FIG. 7 may be a loss function [symbol] for fine-tuning the artificial intelligence model 200.

In the mathematical formula of FIG. 7, θ is the parameter of the artificial intelligence model 200 to be fine-tuned, x_{w} is a preferred molecule, xₗ is a non-preferred molecule, p_{θ} is the probability that the artificial intelligence model 200 to be fine-tuned will assign to a preferred or non-preferred molecule, p_{ref} is the probability that an artificial intelligence model serving as a reference (e.g., the artificial intelligence model 200 before fine-tuning) will assign to a preferred or non-preferred molecule, β is a hyperparameter that adjusts the sensitivity of classification, and P represents the target protein.

The method for fine-tuning an artificial intelligence model that outputs molecules for a protein in accordance with an embodiment of the present disclosure can simultaneously optimize multiple properties that should be considered when developing new drugs by deriving final rewards with a plurality of properties taken into account for the molecules generated by the artificial intelligence model for a target protein.

The method for fine-tuning an artificial intelligence model that outputs molecules for a protein in accordance with an embodiment of the present disclosure can result in stable learning effects through fine-tuning alone without the need for re-training or sampling the artificial intelligence model 200 by utilizing a loss function based on preferences.

Various embodiments of the present specification may be implemented as software (e.g., a program) including one or more instructions stored in a storage medium (e.g., the memory 120) readable by a machine (e.g., the electronic device 100). For example, the processor 110 of the machine (e.g., the electronic device 100) may call upon at least one of the one or more stored instructions from the storage medium and execute it. This enables the machine to operate to perform at least one function according to the at least one called instruction. The one or more instructions may include code generated by a compiler or code executable by an interpreter. The storage medium readable by the machine may be provided in the form of a non-transitory storage medium. Here, “non-transitory” merely means that the storage medium is a tangible device and does not include signals (e.g., electromagnetic waves), and the term does not distinguish between instances where data is stored semi-permanently and instances where data is stored temporarily in the storage medium.

According to one embodiment, the methods in accordance with the various embodiments disclosed herein may be provided in a computer program product. The computer program product may be traded as a commodity between a seller and a buyer. The computer program product may be distributed in the form of a machine-readable storage medium (e.g., compact disc read-only memory (CD-ROM)), or may be distributed online (e.g., downloaded or uploaded) via an application store (e.g., Play Store™) or directly between two user devices (e.g., smartphones). In the case of online distribution, at least part of the computer program product may be at least temporarily stored or temporarily generated in a machine-readable storage medium, such as a memory of a manufacturer's server, an application store's server, or a proxy server.

According to various embodiments, each component (e.g., a module or program) of the components described above may include a single or plurality of entities. According to various embodiments, one or more components or operations of the foregoing components may be omitted, or one or more other components or operations may be added. Alternatively or additionally, a plurality of components (e.g., modules or programs) may be integrated into a single component. In such a case, the integrated component may perform one or more functions of each of the plurality of components in the same or similar manner as performed by the corresponding components of the plurality of components prior to the integration. According to various embodiments, operations performed by modules, programs, or other components may be performed sequentially, in parallel, iteratively, or heuristically, or one or more of the operations may be performed in a different order, or omitted, or one or more other operations may be added.

The above description is merely illustrative of the technical ideas of the present embodiment, and it will be apparent to those of ordinary skill in the art to which the present embodiment pertains that various modifications and variations can be made without departing from the essential characteristics of the present embodiment. Accordingly, the present embodiments are not intended to limit the technical ideas of the present embodiment but to describe them, and the scope of the technical ideas of the present embodiment should not be limited by these embodiments. The scope of protection of the present embodiment should be interpreted based on the following claims, and all technical ideas within the equivalent scope thereof should be interpreted as being included within the scope of rights of the present embodiment.

What is claimed is:

1. An electronic device for fine-tuning an artificial intelligence model that outputs molecules for a protein, comprising:
a processor; and
a memory operatively coupled to the processor,
wherein the memory stores instructions that, when executed, cause the processor to:
identify a molecule suitable for a target protein by inputting the target protein into the artificial intelligence model,
identify a plurality of rewards for each of a plurality of properties required for new drug design, based on the molecule,
identify a final reward for the molecule by normalizing each of the plurality of rewards,
determine a preference for the molecule based on the final reward, and
fine-tune the artificial intelligence model by using a loss function based on the preference.

2. The electronic device of claim 1, wherein the instructions cause the processor to:
scale each of the plurality of rewards,
identify a plurality of normalized values by normalizing each of the scaled plurality of rewards, and
identify the final reward based on the plurality of normalized values.

3. The electronic device of claim 2, wherein the instructions cause the processor to normalize each of the plurality of rewards by using softmax-based normalization.

4. The electronic device of claim 2, wherein the instructions cause the processor to:
identify a mean value for the plurality of normalized values, and
identify the final reward based on the plurality of normalized values and the mean value.

5. The electronic device of claim 4, wherein the instructions cause the processor to identify the final reward based on the plurality of normalized values and the mean value by using uncertainty regularization.

6. The electronic device of claim 4, wherein the instructions cause the processor to identify the final reward based on a deviation between the plurality of normalized values and the mean value.

7. The electronic device of claim 1, wherein the instructions cause the processor to:
receive the molecule suitable for the target protein and a second molecule from the artificial intelligence model, and
determine the preference for the molecule by comparing a second final reward for the second molecule with the final reward for the molecule.

8. The electronic device of claim 1, wherein the plurality of properties comprises at least one of binding affinity between the target protein and the molecule, effectiveness of the molecule, synthetic accessibility, and drug-likeness.

9. An electronic device for fine-tuning an artificial intelligence model that outputs molecules for a protein, comprising:
a processor; and
a memory operatively coupled to the processor,
wherein the memory stores instructions that, when executed, cause the processor to:
identify a molecule suitable for a target protein by inputting the target protein into the artificial intelligence model,
identify a plurality of rewards for each of a plurality of properties required for new drug design, based on the molecule,
identify a final reward for the molecule by normalizing each of the plurality of rewards, fine-tune the artificial intelligence model based on the final reward.

10. The electronic device of claim 9, wherein the instructions cause the processor to:
scale each of the plurality of rewards,
identify a plurality of normalized values by normalizing each of the scaled plurality of rewards, and
identify the final reward based on the plurality of normalized values.

11. The electronic device of claim 10, wherein the instructions cause the processor to normalize each of the plurality of rewards by using softmax-based normalization.

12. The electronic device of claim 10, wherein the instructions cause the processor to:
identify a mean value for the plurality of normalized values, and
identify the final reward based on the plurality of normalized values and the mean value.

13. The electronic device of claim 9, wherein the instructions cause the processor to:
determine a preference for the molecule based on the final reward, and
fine-tune the artificial intelligence model by using a loss function based on the preference.

14. The electronic device of claim 13, wherein the instructions cause the processor to:
receive the molecule suitable for the target protein and a second molecule from the artificial intelligence model, and
determine the preference for the molecule by comparing a second final reward for the second molecule with the final reward for the molecule.

15. A method of fine-tuning an artificial intelligence model that outputs molecules for a protein, performed by an electronic device, comprising the steps of:
identifying a molecule suitable for a target protein by inputting the target protein into the artificial intelligence model;
identifying a plurality of rewards for each of a plurality of properties required for new drug design, based on the molecule;
identifying a final reward for the molecule by normalizing each of the plurality of rewards;
determining a preference for the molecule based on the final reward; and
fine-tuning the artificial intelligence model by using a loss function based on the preference.

16. The method of claim 15, wherein the step of identifying the final reward for the molecule comprises the steps of:
scaling each of the plurality of rewards;
identifying a plurality of normalized values by normalizing each of the scaled plurality of rewards; and
identifying the final reward based on the plurality of normalized values.

17. The method of claim 16, wherein the step of identifying the plurality of normalized values normalizes each of the plurality of rewards by using softmax-based normalization.

18. The method of claim 16, wherein the step of identifying the final reward based on the plurality of normalized values comprises the steps of:
identifying a mean value for the plurality of normalized values; and
identifying the final reward based on the plurality of normalized values and the mean value.

19. The method of claim 18, wherein the step of identifying the final reward based on the plurality of normalized values and the mean value identifies the final reward based on the plurality of normalized values and the mean value by using uncertainty regularization.

20. The method of claim 15, wherein the step of determining the preference for the molecule based on the final reward comprises the steps of:
receiving the molecule suitable for the target protein and a second molecule from the artificial intelligence model; and
determining the preference for the molecule by comparing a second final reward for the second molecule with the final reward for the molecule.
